(19) United States
(12) Patent Application Publication
(10) Pub. No.: US 2026/0013877 A1
(43) Pub. Date: Jan. 15, 2026

(54) REAMING SYSTEM

(71) Applicant: MOWBRAY HOLDINGS LTD., Kamloops (CA)

(72) Inventor: Gordon Robert MOWBRAY, Kamloops (CA)

(21) Appl. No.: 18/875,894

(22) PCT Filed: Jun. 9, 2023
(86) PCT No.: PCT/CA2023/050800

§ 371 Date: Dec. 17, 2024

Related U.S. Application Data
(60) Provisional application No. 63/354,550, filed on Jun. 22, 2022.

(51) Int. Cl. A61B 17/17; A61B 17/00; A61B 17/16

(52) U.S. Cl. CPC A61B 17/1717; A61B 17/1617; A61B 17/1631; A61B 17/164; A61B 2017/00876

(57) ABSTRACT

A reamer system comprising a reamer head and a guidewire. The reamer head defines an elongated slot. The elongated slot extends along a longitudinal axis of the reamer head. In some embodiments, the elongated slot extends an entire length of the reamer head. The guidewire comprises at least two sections, a first section with a first diameter and a second section with a second diameter. The first diameter may be less than the second diameter. In some embodiments, the elongated slot of the reamer head has a width approximately equal to the first diameter of the guidewire. In such embodiments, at least at portion of the first section of the guidewire is receivable within the elongated slot. Means may be provided to rotate the reamer head. Such means may comprise a rotatable drive shaft. In some embodiments, the reamer head is coupleable to a distal end of the drive shaft.

### CROSS-REFERENCE TO RELATED APPLICATION

This application claims priority from U.S. application No. 63/354,550 filed 22 Jun. 2022 and entitled REAMING SYSTEM which is hereby incorporated herein by reference for all purposes. For purposes of the United States of America, this application claims the benefit under 35 U.S.C. § 119 of U.S. application No. 63/354,550 filed 22 Jun. 2022 and entitled REAMING SYSTEM which is hereby incorporated herein by reference for all purposes

### FIELD OF THE INVENTION

The invention pertains to a system for reaming the intramedullary canal of a bone.

### BACKGROUND OF THE INVENTION

Surgical reaming systems are known in the art. During a reaming procedure, surgeons often need to go through multiple rounds of disengagement of reaming heads from the guidewire. Conventional reaming systems require surgeons to disengage a reamer head from the guidewire by pulling a reamer head back the entire length of a guidewire. A guidewire is long, with lengths typically in the range of from 50 to 100 cm. This action is time consuming, undesirably increases procedure time. It is also cumbersome to perform, and can lead to greater risks of contamination and infection. The present invention is directed to improved surgical reaming systems which allow surgeons to disengage reamer heads from a guidewire quickly and easily during the procedure.

### SUMMARY

An aspect of the invention provides a system for reaming an intramedullary canal of a bone. The system comprises a reamer head, a guidewire, and means for rotating the reamer head. In some embodiments, at least a portion of the guidewire arranged to be received within an elongated slot defined along a face of the reamer head. The elongated slot of the reamer head extends along a longitudinal axis of the reamer head. In some embodiments, the elongated slot extends an entire length of the reamer head, from one end of the reamer head to an opposing end thereof. The guidewire comprises a first section defining a first diameter extending to a second section defining a second diameter. In some embodiments, the first diameter is less than the second diameter. In some embodiments, an intermediate section is arranged to extend between the first and second sections. A diameter of the intermediate section tapers from a first end which is in contact with the first section to an opposing second end which is in contact with the second section. At least a portion of the first section and/or the intermediate section is receivable within the slot of the reamer head. The slot of the reamer head is dimensioned to receive at least a portion of the first section of the guidewire.

In some embodiments, the means for rotating the reamer head comprises a drive shaft. In some embodiments, the reamer head is coupleable to a drive shaft. The drive shaft has a proximal end and an opposing distal end. The reamer head may be coupleable to the distal end of the drive shaft so as to drive a rotation of the reamer head.

In some embodiments of the invention, first and second magnetized zones are arranged on the reamer head and the drive shaft respectively. In example embodiments, the first magnetized zone is arranged on a head connector of the reamer head and the second magnetized zone is arranged on a shaft connector of the drive shaft. The first and second magnetized zones may be arranged to strengthen the connection between the reamer head and the drive shaft.

In some embodiments, a finger is arranged to protrude outwardly from the distal end of the drive shaft. The finger is dimensioned to be received within the elongated slot of the reamer head. The finger may be provided to avoid compression of the reamer head during use by filling the opening of the elongated slot.

Further aspects of the invention and features of specific embodiments of the invention are described below.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a partial cutaway view of an example surgical reaming system comprising a drive shaft, a reaming head and a guidewire according to a first example embodiment.

FIG. 2 is a perspective view of the FIG. 1 surgical reaming system illustrating the reaming head and the guidewire.

FIG. 3 is a cross-sectional view of the FIG. 1 reaming head.

FIG. 4 is a schematic view of the FIG. 1 guidewire.

FIG. 5 is a plan view of the FIG. 1 guidewire.

FIG. 6 is a perspective view of a drive shaft of a surgical reaming system according to a second example embodiment.

### DETAILED DESCRIPTION

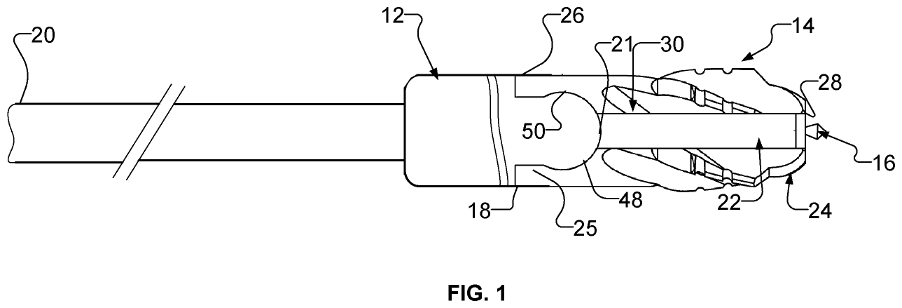

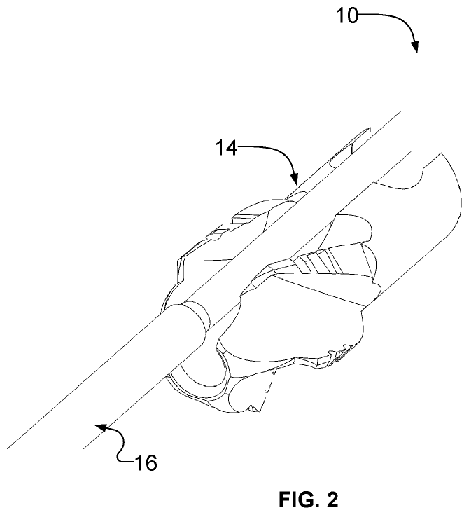

The invention provides a system for reaming an intramedullary canal of a bone. Referring to FIGS. 1-5, the system 10 comprises a reamer head 14, and a guidewire 16. Means may be provided to rotate the reamer head 14. In some embodiments, such means comprises a rotatable drive shaft 12. The rotatable drive shaft 12 may be coupleable to the reamer head. In some embodiments, the rotatable drive shaft 12 comprises a proximal end 20 and an opposing distal end 18. The proximal end 20 is adapted to connect to a drive element (not shown) to rotate the drive shaft 12. The drive element may be a motorized handpiece such as but not limited to a drill. In some embodiments, the proximal end 20 of the drive shaft 12 connects to the drive element via an attachment piece (not shown). Such attachment piece may for example be an AO or a trinkle drill attachment. The drive shaft 12 may terminate in a shaft connector 21 at the distal end 18.

In some embodiments, the reamer head 14 is coupleable to the distal end 18 of the drive shaft 12. The reamer head 14 defines an elongated slot 22 along an outer face 24 of the reamer head 14, and extends along a longitudinal axis of the reamer head 14. In some embodiments, the slot 22 extends an entire length of the reamer head 14. In such embodiments, the slot 22 extends along a longitudinal axis of the reamer head 14 from a first end 26 of the reamer head 14 to an opposing second end 28 of the reamer head 14. The slot 22 may define a width, measured by a distance between opposing points on the inner face 30 of the slot. In some embodiments, the width is defined by a distance from one point on the inner face 30 through to a center of the slot 22, therefrom to a second point on the inner face 30 of the slot 22. In some embodiments, the width of the slot 22 is the diameter of the slot 22. The width of the slot 22 may be varied depending on the diameter of the guidewire 16. The width of the slot 22 is sized to receive a portion of the guidewire 16. The width of the slot 22 may be in the range of from 1 mm to 5 mm, including for example 2 mm to 4 mm. In one non-limiting example embodiments in which the guidewire 16 has a maximum diameter of about 3 mm which tapers to about 2 mm, the width of the slot 22 is about 2 mm, or 2 mm+/−0.1 mm to 0.5 mm, and less than 3 mm.

In some embodiments, the guidewire 16 is arranged to extend through a channel (not shown) which is defined within the drive shaft 12 in a longitudinal axis thereof, and through the slot 22 of the reamer head 14. The guidewire 16 extends outwardly through the slot 22 from the opposing second end 28 of the reamer head 14.

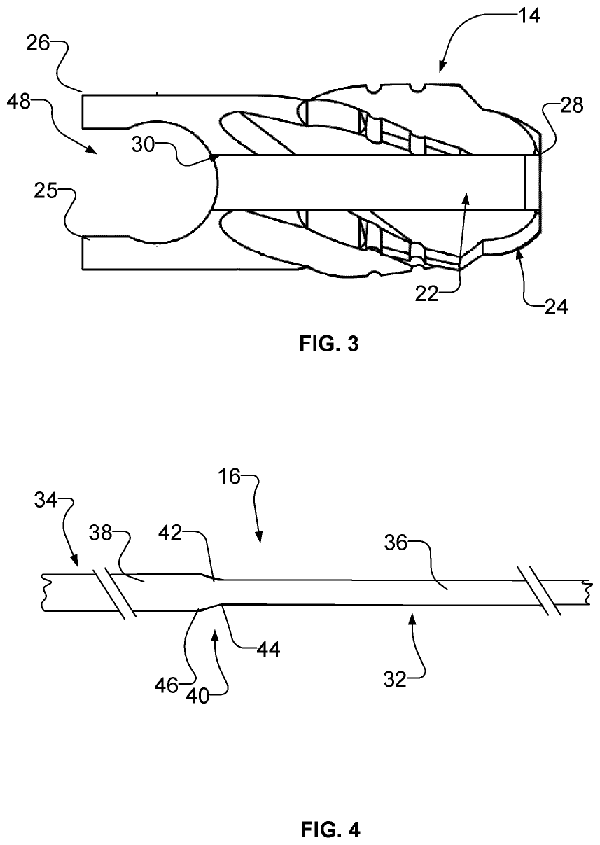

As best shown in FIG. 3, the reamer head 14 may comprise a head connector 25 at the first end 26. The head connector 25 may be dimensioned to engage with a shaft connector 21 for securing the reamer head 14 to the drive shaft 12. In example embodiments, the reamer head 14 and the drive shaft 12 are connected by a dovetail joint. In some example embodiments, the head connector 25 comprises a female dovetail connection, and the shaft connector 21 comprises a male dovetail connection.

Means may be provided to strengthen the connection between the reamer head 14 and the drive shaft 12. In an example embodiment, the strengthener means includes one or more magnetized zones. In some embodiments, first and second magnetized zones 48, 50 may be arranged on the reamer head 14 and the drive shaft 12 respectively to strengthen the connection therebetween, thereby preventing premature disengagement between the two components. In some embodiments, the first magnetized zone 48 is secured to the head connector 25, and the second magnetized zone 50 is secured to the shaft connector 21.

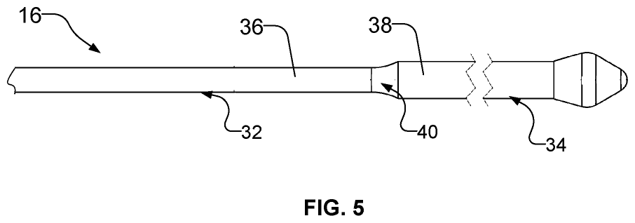

At least a portion of the guidewire 16 is arranged to be received within the elongated slot 22 of the reamer head 14. As best shown in FIGS. 4 and 5, the guidewire 16 may comprise a first section 32 extending to a second section 34 to form an entire length of the guidewire 16. The first section 32 is defined by a first diameter 36, and the second section 34 is defined by a second diameter 38. In some embodiments, the first diameter 36 is less than the second diameter 38. In example embodiments, the first diameter 36 is approximately ⅔ to ¾ of the second diameter 38. In one non-limiting example embodiment, the first diameter 36 is approximately ⅔ of the second diameter 38. For example, in such embodiment, the first diameter 36 may be 2 mm and the second diameter 38 may be 3 mm.

In some embodiments, an intermediate section 40 extends between the first and second sections 32, 34 of the guidewire 16. In an example embodiment, the intermediate section 40 is positioned at a midpoint of the entire length of the guidewire 16. A diameter 42 of the intermediate section 40 may taper along its length, from a first end 46 which is in contact with the second section 34 to a second opposing end 44 which is in contact with the second section 32. In one non-limiting example embodiment in which the first diameter 36 is 2 mm and the second diameter 38 is 3 mm, the diameter 42 of the intermediate section 40 may taper from 3 mm to 2 mm along its length, from the first end 46 to the second end 44. In some embodiments, the length of the intermediate section 40 is about 0.5% to about 5% of the entire length of the guidewire 16. In example embodiments, the length of the intermediate section 40 is about 1% to about 2% of the entire length of the guidewire 16. In one example embodiment, the entire length of the guidewire 16 is 60 cm and the length of the intermediate section 40 is 10 mm. In another example embodiment, the entire length of the guidewire 16 is 100 cm and the length of the intermediate section 40 is 10 mm.

In some embodiments, the first and second sections 32, 34 have different lengths. In some embodiments, first and second sections 32, 34 are approximately equal in length.

At least a portion of the first section 32 of the guidewire 16 is arranged to be received within the elongated slot 22 of the reamer head 14. In some embodiments, an entire length of the first section 32 of the guidewire 16 is received within the elongated slot 22.

The first diameter 36 of the first section 32 of the guidewire 16 is dimensioned to be received within the slot 22 of the reamer head 14. In some embodiments, the first diameter 36 and the width of the slot 22 are approximately equal so as to allow the first section 32 to snuggly fit within the slot 22. For example, in one embodiment, both the first diameter 36 and the width of the slot 22 are about 2 mm. In some embodiments, the first diameter 36 and the width of the slot 22 have values within +/−2% to 10% one of another.

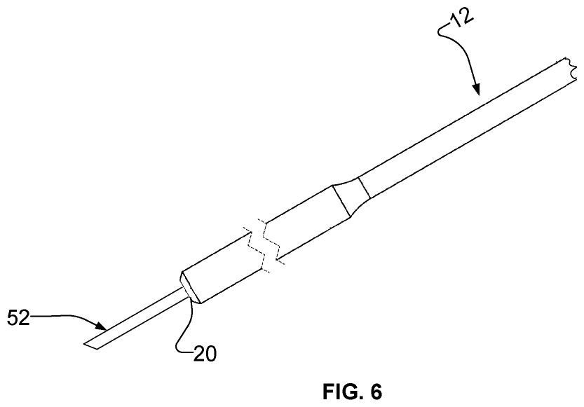

Referring to FIG. 6, in some embodiments, a finger 52 is arranged to protrude outwardly from the distal end 18 of the drive shaft 12. The finger 52 is dimensioned to be insertable within the slot 22 of the reamer head 14 along a longitudinal axis of the reamer head 14. In some embodiments, a width of the finger 52 and the width of the slot 22 are approximately equal. In some embodiments, the width of the finger 52 and the width of the slot 22 have values within +/−2% to 10% of one another. The entire length of the finger 52 may be approximately equal to the entire length of the slot 22. In some embodiments, the entire length of the finger 52 and the entire length of the slot 22 have values within +/−2% to 10% of one another. The finger 52 may be provided to avoid compression of the reamer head 14 during use by filling the opening of the slot 22.

During a reaming procedure, surgeons often need to use multiple reaming heads of gradually increasing diameter until the desired reamed size is achieved. This requires surgeons to go through multiple rounds of disengagement of reaming heads from the guidewire during one procedure. With conventional reaming systems, surgeons will need to pull a reamer head back the entire length of a guidewire before the reamer head can be disengaged from the guidewire. This is time consuming, undesirably increases procedure time, and is cumbersome to perform which can result in greater risks of contamination and infection. The system 10 of this invention advantageously allows surgeons to disengage the reamer head 14 from the guidewire 16 quickly and easily. In use, the reamer head 14 can be disengaged from the guidewire 16 by pulling the reamer head 14 to a point along the first section 32 of the guidewire 16, which in some embodiments, may be roughly the midpoint of the guidewire 16. The reamer head 14 can therefore be disengaged from the guidewire 16 at roughly the midpoint of the guidewire 16 rather than having to pass the reamer head over the entire length of the guidewire as would be required when using conventional reaming systems.

Throughout the foregoing description and the drawings, in which corresponding and like parts are identified by the same reference characters, specific details have been set forth in order to provide a more thorough understanding to persons skilled in the art. However, well known elements may not have been shown or described in detail or at all to avoid unnecessarily obscuring the disclosure.

As will be apparent to those skilled in the art in the light of the foregoing disclosure, many alterations and modifications are possible in the practice of this invention without departing from the scope thereof. Accordingly, the description and drawings are to be regarded in an illustrative, rather than a restrictive, sense

1. A reaming system (10) comprising:
a reamer head (14) having an outer face (24) defining an elongated slot (22), the elongated slot extending along a longitudinal axis of the reamer head; and
a guidewire (16) comprising a first section (32) extending to a second section (34), wherein a diameter (36) of the first section is less than a diameter (38) of the second section,
wherein the reamer head is slideable along the guidewire between the first and second sections of the guidewire,
and wherein the reamer head is disengageable from the guidewire when at least a portion of the first section of the guidewire is positioned in the elongated slot, and the portion of the first section of the guidewire is positioned between opposing ends of the reamer head.

2. The reaming system according to claim 1, further comprising means (12) for rotating the reamer head.

3. The reaming system according to claim 2, wherein the means for rotating the reamer head comprises a rotatable drive shaft (12) coupleable to the reamer head.

4. The reaming system according to claim 3, the rotatable drive shaft comprises proximal (20) and distal (18) ends, wherein the proximal end is adapted to connect to a drive element to rotate the drive shaft.

5. The reaming system according to claim 1, wherein the slot of the reamer head has a width approximately equal to the diameter of the first section of the guidewire.

6. The reaming system according to claim 1, wherein the diameter of the first section is about ⅔ to ¾ of the diameter of the second section.

7. The reaming system according to claim 1, wherein the diameter of the first section is about ⅔ of the diameter of the second section.

8. The reaming system according to claim 1, wherein the guidewire further comprises an intermediate section (40) joining the first section to the second section.

9. The reaming system according to claim 8, wherein a diameter (42) of the intermediate section tapers along a length thereof, from an end (46) in contact with the second section to an opposing end (44) in contact with the first section.

10. The reaming system according to claim 1, wherein the first and second sections are approximately equal in length.

11. The reaming system according to claim 1, further comprising first (48) and second (50) magnetized zones arranged on the reamer head and the distal end of the drive shaft respectively.

12. The reaming system according to claim 1, wherein the slot extends along an entire length of the reamer head from one end (26) to an opposing end (28) thereof.

13. The reaming system according to claim 1, further comprising a finger (52) receivable within the slot of the reamer head, protruding from the distal end of drive shaft.

14. The reaming system according to claim 13, wherein a width of the finger is approximately equal to the width of the slot of the reamer head.
